(19) United States
(12) Patent Application Publication
(10) Pub. No.: US 2015/0044316 A1
(43) Pub. Date: Feb. 12, 2015

(54) METHOD FOR PREPARING A PURIFIED EXTRACT OF LONICERA JAPONICA THUNBERG AND THE COMPOSITION COMPRISING THE SAME FOR PREVENTING AND TREATING SEPSIS AND SEPTIC SHOCK

(71) Applicant: Huons Co., Ltd., Seongnam-Si (KR)

(72) Inventors: Sung-Tae Yoon, Seoul (KR); Jeong Hoon Kim, Seongnam-Si (KR); Bang Ho Lim, Seoul (KR); Young Mok Kim, Seoul (KR); Sung Hum Yeon, Hwaseong-si (KR); Hyun Soo Kim, Changwon-si (KR); Sun-Mee Lee, Seoul (KR)

(21) Appl. No.: 14/397,468

(22) PCT Filed: Nov. 1, 2012
(86) PCT No.: PCT/KR2012/009100

§ 371 Date: Oct. 27, 2014

(30) Foreign Application Priority Data
Apr. 27, 2012 (KR) 10-2012-0044441

(51) Int. Cl. A61K 36/355; A23L 1/30; A61K 45/06

(52) U.S. Cl. CPC A61K 36/355; A61K 45/06; A23L 1/3002
USPC 424/778

(57) ABSTRACT

The present invention relates to a method for preparing a purified extract of Lonicera Japonica THUNBERG and the composition comprising the same for preventing and treating sepsis and septic shock. The purified extract of purified extract of Lonicera Japonica THUNBERG potent antisepsis activity in severe sepsis CLP model test, the effect on MODS, and the inhibitory effect on various pro-inflammatory cytokines such as TNF-alpha, IL-1beta, IFN-gamma, HMGB-1 etc, as well as it showed unexpectedly synergistic effect on the treatment of sepsis and septic shock in case of combining with the commercially available anti-septic agent such as broad-spectrum anti-biotic to the person skilled in the art, therefore, it can be useful in treating and preventing the sepsis and septic shock as a medicament and health functional food.

### TECHNICAL FIELD

The present invention relates to a method for preparing a purified extract of Lonicera Japonica THUNBERG and the composition comprising the same for preventing and treating sepsis and septic shock.

### BACKGROUND ART

Severe sepsis is fatal disease with high mortality caused by septic condition accompanying with organ function failure and hypo-perfusion resulting in the unbalance between systemic oxygen demand and oxygen supply, which rapidly develops to septic shock and multiple organ dysfunction syndrome (MODS). The representative syndromes of the disease are high fever, hypothermia, positive chronotrophy, increased cardiac output, decreased resistance on systemic circulation, respiratory alkalosis, abnormally increased or decreased number of WBC etc, which causes to rapid organ dysfunction resulting in fatal death. The mortality rate of the patients suffering with severe sepsis and septic shock has been reported to 29% in U.S.A and 27% in Europe, which is a main cause of death among the patients in intensive care unit (Jean-Louis Vincent, Edward Abraham, The Last 100 years of Sepsis, Am J Respir Crit Care Med, 2006, 173, pp 256-263). In U.S.A, more than 750,000 patients/year are suffered with septic syndrome and more than 210,000 patients/year among them die with the disease. Moreover, approximately 37% patients and 15% patients being hospitalized at intensive care unit in Europe suffer with severe sepsis and septic shock respectively and the mortality rate caused by severe sepsis is 65% in Korea.

Sepsis may be deteriorated by the non-communicable origins such as scar besides the communicable origins such as germ, viruses, fungi etc and exacerbated to septic shock or MODS (Deitch, E. A. Multiple organ failure. Pathophysiology and potential future therapy, Ann. Surg., 1992, 216(2), pp. 117-134). The most apparent characteristic of the sepsis pathogenesis is the hyper-activation of inflammatory system in the body at the initial stage, i.e., the hyper-secretion of the pro-inflammatory cytokines recognizing and activated by foreign contaminants such as bacterial endo-toxin, which is called as “cytokine storm” and maintained for 10-12 hrs from the onset of sepsis. Among the cytokines, TNG-alpha, a cytokine detected at the first of initial stage in the blood, not only stimulates the secretion of other cytokines such as IL-5, IL-8, but also promotes the expression of cell-adhesive molecules in neutrophils and vascular endothelial cells together with other cytokines resulting in the exacerbation of inflammatory response (Wada H. et al., Increased plasma level of interleukin-6 in disseminated intravascular coagulation, Blood Coagul. Fibrinolysis, 1993, 4(4); p 583-590; Qin, S. et al., Role of HMGB1 in apoptosis-mediated sepsis lethality, J. Exp. Med., 2006, 203(7), pp. 1637-1642). IL-6, an inflammatory cytokine secreted from lymphocytes and monocytes, reached to the highest level at 6 hours after the onset of sepsis (Hotchkiss, R. S., et al., Apoptosis and caspases regulate death and inflammation in sepsis. Nat. Rev. Immunol., 2006, 6(11), pp 813-822).

Accordingly, there have been lots of attempts to inhibit the inflammatory mediators accompanying and stimulating the inflammation such as TNF-alpha, IL-6, IL-1, IL-8 and the like, for examples, anti-inflammatory agents such as NSAIDS; or to inhibit the release of cytokines, for example, a TLR4 selective inhibitor such as TAK-242® from Takeda Pharm. Company Limited, and Eritoran® from Eisai Co. Ltd. However the therapy has several disadvantages such as limited efficacy and the like.

Xigris® (Eli lilly and company), a sole treating agent to severe sepsis approved from U.S.A and Europe recently, has been prescribed to treat severe sepsis however it also has the disadvantages, for example, limited indication and efficacy, adverse response such as severe hemorrhage or stroke etc.(R. Phillip Dellinger etal., Important issues in the design and reporting of cinical trilas in severe sepsis as acute lung injury, Journal of Critical Care.,23 pp 493-499, 2008; www.fda.gov.)

Accordingly, there have been still needed to develop new anti-sepsis drug with potent efficacy and less toxicity till now and tried to develop effective drugs from natural resource which has been frequently prescribed and used due to its non-toxicity in Korea.

The flower of Lonicera Japonica THUNBERG distributed in Korea has been reported to comprise luteolin, inositol, saponin, tannins, isochlorogenic acod, chlorogenic acid etc (B. S. CHUNG et al, Dohaehyangyakdaesajeon, Youngrim press, pp. 939-940, 1998).

However, there has been not reported or disclosed about the therapeutic effect or improving effect for sepsis of the purified flower extract of Lonicera Japonica THUNBERG in any of above cited literatures, the disclosures of which are incorporated herein by reference.

To investigate the novel method for preparing purified flower extract of Lonicera Japonica THUNBERG comprising abundant amount of active ingredient such as chlorogenic acid and the derivatives thereof and the treating effect of the purified flower extract of Lonicera Japonica THUNBERG on sepsis disease, the inventors of the present invention have intensively carried out component analysis as well as several animal model tests such as severe sepsis CLP model test, the effect on MODS, the inhibitory effect on various pro-inflammatory cytokines such as TNF-alpha, IL-1beta, IFN-gamma, HMGB-1 etc, and finally completed present invention by confirming that the purified extract of the flower extract comprise abundant amount active ingredient and show potent treating effect on sepsis and septic shock.

These and other objects of the present invention will become apparent from the detailed disclosure of the present invention provided hereinafter.

### DISCLOSURE OF INVENTION

### Technical Problem

Accordingly, it is an object of the present invention to provide a method for preparing a purified extract of Lonicera Japonica THUNBERG comprising the step consisting of; extracting the dried flower material of Lonicera Japonica THUNBERG with extracting solvent at 1^{st} step; subjecting the crude extract to at least one treatment selected from filtration method, centrifugation or the combination thereof, preferably, filtration method to afford the crude extract of Lonicera Japonica THUNBERG at 2^{nd} step; suspending the crude extract in water by adding water to prepare the suspended solution and fractionating the solution into non-polar solvent soluble fraction and polar solvent soluble fraction to remove the non-polar soluble substance and to afford the 1^{st} purified extract by collecting the residue at 3^{rd} step; adding water to the 1^{st} purified extract to subject to at least one purification process selected from adsorption chromatography, ion column chromatography or the combination thereof using by the equivalent amount of adsorbent resin to that of water, and washing with washing solvent repeatedly to afford the 2^{nd} purified extract of Lonicera Japonica THUNBERG at 4^{th} step; and concentrating the extract under vaccuo and drying to afford the purified extract of Lonicera Japonica THUNBERG containing abundant amount of active ingredients, specifically, chlorogenic acid and the derivatives thereof in an amount ranging from 2.0 to 30.0%(w/w), preferably, 5.0 to 20.0% (w/w), more preferably 7.0 to 15.0% (w/w) based on the weight of the dried purified extract.

In a preferred embodiment of the present invention, the extracting solvent at 1^{st} step in the above-described method, comprises approximately 1 to 100 fold, preferably, 2 to 20 fold, more preferably, 5 to 15 fold volume of at least one solvent based on the weight of flower material (v/w) selected from the group consisting of water, spirit, methanol, ethanol, propanol, butanol, hexane, ethylacetate, cyclohexane, DMSO, chloroform and methylene chloride, preferably, the group of water, methanol, ethanol, propanol and, butanol, more preferably, water, most preferably, basic solution dissolving weak base such as NaHCO₃, NaCO₃ etc in an amount of 0.1 to 5%, preferably, 0.2 to 2% weight based on the weight of the flower material (w/w) in water to improve the extraction efficiency.

In a preferred embodiment of the present invention, the extracting process at 1^{st} step in the above-described method, is performed by at least one extraction method selected from hot-water reflux extraction, enfleurage extraction, Soxhlet extraction, sonication extraction and the combination thereof, preferably, hot-water reflux extraction at the temperature ranging from 20 to 120° C., preferably, 30 to 100° C., for the period ranging from about 1 to 72 hours, preferably, 2 to 12 hours.

In a preferred embodiment of the present invention, the treatment to afford the crude extract of Lonicera Japonica THUNBERG at 2^{nd} step in the above-described method, is performed by at least one treatment selected from filtration method, centrifugation or the combination thereof, preferably, filtration method.

In a preferred embodiment of the present invention, the process to afford the 1st purified extract process at 3rd step in the above-described method, is performed by adding about 0.005 to 5 fold volume, preferably, 0.05 to 3 fold volume of water (v/w, based on the weight of the crude extract) to prepare the suspended solution; and fractionating the solution into non-polar solvent soluble fraction and polar solvent soluble fraction to remove the non-polar soluble substance by adding about 0.1 to 50 fold volume, preferably, 0.5 to 10 fold volume of non-polar solvent (v/v, based on the volume of the suspension) such hexane, methylene chloride, chloroform, ethyl acetate etc, preferably, hexane, methylene chloride, or ethyl acetate, more preferably, hexane or methylene chloride.

Through the purification process at 3^{rd} step, the non-polar soluble substance in the extract, for example, essential oils such as hexadecanoic acid, methyl linolate, linalool, carvacrol, methyl palmitate etc and sterol compounds such as beta sitosterol etc can be efficiently removed from the extract.

In a preferred embodiment of the present invention, the process to afford the 2^{nd} purified extract of Lonicera Japonica THUNBERG at 4^{th} step in the above-described method, is performed by adding about 1 to 30 fold weight, preferably, 2 to 15 fold weight, more preferably, 5 to 10 fold weight of water (w/w, based on the weight of the 1^{st} purified extract) to the 1^{st} purified extract to subject to adsorption chromatography using by the equivalent amount of adsorbent resin to that of water, preferably, at least one resin selected from SP207, HP20SS, Diaion HP 20, SP-850 resin, active carbon, or Amberlite XAD-2,4, more preferably, at least one resin selected from Diaion HP 20, SP-850 resin or Amberlite XAD-2,4 for further purification.

In a preferred embodiment of the present invention, the process to afford the 2nd purified extract of Lonicera Japonica THUNBERG at 4th step in the above-described method, is performed by subjecting to ion column chromatography using by the equivalent amount of ionic resin to that of water, for example, at least one strongly acidic resin selected from AG 50W-x8, Amberlite IR-120, Amberlite IRA-400, Dowex 50W-x8 or SK1B; at least one weakly acidic resin selected from Amberlite IRC-50, Bio-Rex 70, Duolite-436 or WK40; or at least one weakly basic resin selected from Amberlite IR-67 or Dowex 3-x4, preferably, at least one strongly acidic resin selected from Amberlite IR-120, Amberlite IRA-400 or SK1B, more preferably, at least one strongly acidic resin selected from Amberlite IR-120, or Amberlite IRA-400.

In a preferred embodiment of the present invention, the washing process to afford the 2^{nd} purified extract of Lonicera Japonica THUNBERG at 4^{th} step in the above-described method, is performed by washing the adsorbent to the resin with at least on washing solvent selected from water, methanol, ethanol, propanol, butanol or the mixture thereof, preferably, the mixture solvent with water and methanol, repeatedly.

Through the purification process at 4th step, the inactive substance showing no pharmacological activity, for example, amino acid such as proline etc and sugars such as glucose, sucrose, inositol etc can be efficiently removed from the extract.

In an alternative embodiment of the present invention, it is preferable to perform further purification process, for example, sephadex column chromatography using by at least on sephadex resin selected from Sephadex LH-20 resin, Sephadex G15 resin or Sephadex G35 and the like besides the purification process at 4^{th} step in the above-described method.

In a preferred embodiment of the present invention, the concentrating process and drying process to afford the purified extract of Lonicera Japonica THUNBERG at 5^{th} step in the above-described method, is performed by concentrating the extract under vaccuo at the temperature ranging from 10 to 80° C., preferably, less than 60° C. and drying the extract by at least one drying method selected from room temperature drying method, freeze drying method, hot-air drying method or the combination thereof, preferably, freeze drying method to afford inventive purified extract of extract of Lonicera Japonica THUNBERG (designated as “HS-23 extract”, hereinafter).

Through the inventive purification process in the above, the inventive inventors have found that the inventive purified extract of Lonicera Japonica THUNBERG prepared by the above-described method, contains abundant amount of active ingredients, specifically, about 5.8 fold yield of chlorogenic acid and about 5.2 fold yield of the total chlorogenic acid derivatives including chlorogenic acid and the its derivatives, for example, 3,5-O-caffeoylquinic acid, methyl 3,5-di-O-caffeoyl quinate and the like in an amount ranging from 2.0 to 30.0% (w/w), preferably, 5.0 to 20.0% (w/w), more preferably 7.0 to 15.0% (w/w) based on the weight of the dried purified extract, of which yield is more than 5 folds than that prepared by the well-known extraction method for preparing the extract of Lonicera Japonica THUNBERG in the art.

Moreover, the HS extract showed potent ant-sepsis activity in severe sepsis CLP model test, the effect on MODS, and the inhibitory effect on various pro-inflammatory cytokines such as TNF-alpha, IL-1beta, IFN-gamma, HMGB-1 etc, as well as it showed unexpectedly synergistic effect on the treatment of sepsis and septic shock in case of combining with the commercially available anti-septic agent such as broad-spectrum anti-biotic (about 120% increased survival rate than the sole treatment group in severe sepsis induced animal model) to the person skilled in the art.

### Solution to Problem

Accordingly, it is an object of the present invention to a purified HS-23 extract of Lonicera Japonica THUNBERG containing chlorogenic acid and the derivatives thereof in an amount ranging from 2.0 to 30.0% (w/w), preferably, 5.0 to 20.0% (w/w), more preferably 7.0 to 15.0% (w/w) based on the weight of the dried purified extract, which is prepared by the step consisting of; extracting the dried flower material of Lonicera Japonica THUNBERG with extracting solvent at 1^{st} step; subjecting the crude extract to at least one treatment selected from filtration method, centrifugation or the combination thereof, preferably, filtration method to afford the crude extract of Lonicera Japonica THUNBERG at 2^{nd} step; suspending the crude extract in water by adding water to prepare the suspended solution and fractionating the solution into non-polar solvent soluble fraction and polar solvent soluble fraction to remove the non-polar soluble substance and to afford the 1^{st} purified extract by collecting the residue at 3^{rd} step; adding water to the 1^{st} purified extract to subject to at least one purification process selected from adsorption chromatography, ion column chromatography or the combination thereof using by the equivalent amount of adsorbent resin to that of water, and washing with washing solvent repeatedly to afford the 2^{nd} purified extract of Lonicera Japonica THUNBERG at 4^{th} step; and concentrating the extract under vaccuo and drying to afford the purified extract of Lonicera Japonica THUNBERG for the treatment and prevention of sepsis, MODS or septic shock.

Accordingly, it is an object of the present invention to provide a pharmaceutical composition comprising the purified HS-23 extract of Lonicera Japonica THUNBERG purified by the above-described method as an active ingredient for the treatment and prevention of sepsis, MODS or septic shock.

The inventive purified HS-23 extract having more potent pharmacological effect than the extract prepared by the well-known extraction method, may comprise chlorogenic acid and the derivatives thereof in an amount ranging from 2.0 to 30.0% (w/w), preferably, 5.0 to 20.0% (w/w), more preferably 7.0 to 15.0% (w/w) based on the weight of the dried purified extract.

It is an object of the present invention to provide a use of the purified HS-23 extract of Lonicera Japonica THUNBERG purified by the above-described method for the preparation of therapeutic agent for the treatment and prevention of sepsis, MODS or septic shock in mammal including human.

It is an object of the present invention to provide a method of treating or preventing sepsis, MODS or septic shock in mammal including human comprising administering an effective amount of the purified HS extract of Lonicera Japonica THUNBERG purified by the above-described method, together with a pharmaceutically acceptable carrier thereof to said mammal.

The term “sepsis” disclosed herein comprises various sepsis, but not intended to limit to herein, for example, mild sepsis, severe sepsis, infection symptoms or sepsis caused by burn, acute laryngopharyngitis, ulcerative colitis, IBS (Irritable Bowel syndrome), rheumatic arthritis, degenerative arthritis, acute hepatitis, chronic hepatitis, etc, preferably, mild sepsis, severe sepsis, infection symptoms or sepsis caused by burn.

The term “MODS (multiple organ dysfunction syndrome)” disclosed herein comprises various MODS occurs, but not intended to limit to herein, for example, in the injured organ selected from liver, kidney, heart, lung, small intestine, large intestine, duodenum, stomach, pancreas, spleen, etc, preferably, liver, kidney, or heart caused by mild sepsis, severe sepsis, or infection symptoms or sepsis caused by burn, preferably severe sepsis.

The term “Septic shock” disclosed herein comprises various septic shock, but not intended to limit to herein, for example, septic shock caused by mild sepsis, severe sepsis, infection symptoms or sepsis caused by burn.

The HS extract showing potent ant-sepsis activity can be combined with the commercially available anti-septic agent in order to obtaining synergistic effect to treat and prevent sepsis, MODS or septic shock.

Accordingly, it is an another object of the present invention to provide a pharmaceutical composition comprising the combination of purified HS-23 extract of Lonicera Japonica THUNBERG purified by the above-described method with the commercially available anti-septic agent as an active ingredient for the treatment and prevention of sepsis, MODS or septic shock.

It is an object of the present invention to provide a use of the combination of purified HS-23 extract of Lonicera Japonica THUNBERG purified by the above-described method with the commercially available anti-septic agent for the preparation of therapeutic agent for the treatment and prevention of sepsis, MODS or septic shock in mammal including human.

It is an object of the present invention to provide a method of treating or preventing sepsis, MODS or septic shock in mammal including human comprising administering an effective amount of the combination of purified HS-23 extract of Lonicera Japonica THUNBERG purified by the above-described method with the commercially available anti-septic agent, together with a pharmaceutically acceptable carrier thereof to said mammal.

The term “the combination of purified HS-23 extract of Lonicera Japonica THUNBERG purified by the above-described method with the commercially available anti-septic agent” disclosed herein comprises the combination of (a) purified HS extract of Lonicera Japonica THUNBERG purified by the above-described method with (b) the commercially available anti-septic agent mixed with the mixed ratio from 0.1˜10:0.1˜10 by weight (w/w), preferably, 1˜10:1˜10 by weight (w/w), more preferably, 1˜5:1˜5 by weight (w/w).

The term “the commercially available anti-septic agent” disclosed herein comprises various commercially available anti-septic agent, but not intended to limit to herein, for example, at least one anti-septic agent selected from the group consisting of antibiotics such as penicillin, quinolone, monobactam, aminoglycoside, cephalosporin, tetracycline, glycopeptides, carbapenem and the like; anti-inflammatory agents such as mefenamic acid, indomethacin, ibuprofen, piroxicam, diclofenac and the like; antifungal agent such as amphotericin, B, nystatin, griseofulvin, azole anti-fungal agent and the like; and anti-allergic agent such as cetirizine, fexofenadine, chlroropeniramine, and the like, preferably, anti-septic agent selected from the group consisting of antibiotics such as penicillin, quinolone, monobactam, aminoglycoside, cephalosporin, tetracycline, glycopeptides, carbapenem and the like; more preferably, anti-septic agent selected from the group consisting of amoxicillin, ampicillin, vancomycin, amikacin, imipenam and the like;

The pharmaceutical composition of the present invention can contain about 0.01˜50% by weight of the above extract based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

Pharmaceutical formulations containing present composition may be prepared in any form, for example, oral dosage form such as lyophilized preparation, powder, granule, tablet, capsule, soft capsule, elixirs pill, sachet etc as a solid oral formulation; suspension, solution, emulsion, syrup, aqueous medicine etc as a liquid oral formulation; topical preparation such as cream, ointment, lotion, gel, balm, patch, paste, spray solution, aerosol and the like; or parenteral dosage forms, for example, suppositories or injectable preparation such as sterilized solution, suspension, lyophilized preparation, non-aqueous type injection, or aqueous type injection, preferably, sterilized injectable preparation.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include solvent, additive, diluents, buffer, isotonic agent, stabilizer, anti-oxidant, pain-reliever, emulsifier, fillers, anti-agglutinating agents, lubricating agents, wetting agents, flavoring agents, preservatives etc. Specifically, said solvent, additive, or diluents includes sterilized distilled water, physiological saline solution, pH controller, albumin, sodium chloride, mannitol, Ringer's solution, glucose etc. The solid oral formulation such as powder, granule, tablet, capsule, soft capsule, elixirs pill, sachet etc may be prepared by mixing the inventive extract with at least one adjuvant, for example, starch, calcium carbonate, sucrose, lactose, gelatin etc, if necessary, lubricants such as magnesium stearate, talc etc as a additional additive to be formulated. The liquid oral formulation such as suspension, solution, emulsion, syrup, aqueous medicine etc may be prepared by mixing the inventive extract with at least one adjuvant, for example, wetting agent, flavoring agent, sweetener, preservative, other than common diluents such as water or liquid paraffin to be formulated. As the parenteral dosage forms, for example, injectable preparation such as sterilized solution, suspension, lyophilized preparation, non-aqueous type injection, or aqueous type injection, may use propylene glycol, polyethylene glycol, vegetable oil such as olive oil, injectable ester such as ethyl olate etc as a base; and suppositories may use whitepsol, macrogol, tween 61, cacao oil, lauric oil, glycerol-gelatin etc as a base in the present invention.

The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging from 1 microgram to 5 mg/day, preferably, 8 microgram to 2 mg/day, more preferably, 16 microgram to 1 mg/day of the inventive extract of the present invention. The dose may be administered in single or divided into several times per day; periodically, for example, once for a period ranging from 2 days to one week, but are not intended to limit thereto. The scope of present invention may include all the modification, or change in terms of any amount and number of dosage, and any administration pathway which can be conceivable by the artisan in the art. In terms of composition, the amount of inventive extract may be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Inventive extract of the present invention have no toxicity and adverse effect therefore; they can be used with safe.

The present invention provides a health functional food comprising purified HS-23 extract of Lonicera Japonica THUNBERG purified by the above-described method as an active ingredient for alleviating or preventing sepsis, MODS or septic shock.

The present invention provides a health functional food comprising the combination of purified HS-23 extract of Lonicera Japonica THUNBERG purified by the above-described method with the commercially available anti-septic agent as an active ingredient for alleviating or preventing sepsis, MODS or septic shock.

The present invention also provides a health functional food comprising the purified HS-23 extract of Lonicera Japonica THUNBERG purified by the above-described method, and a sitologically acceptable additive for alleviating or preventing sepsis, MODS or septic shock.

The present invention also provides a health functional food comprising the combination of purified HS-23 extract of Lonicera Japonica THUNBERG purified by the above-described method with the commercially available anti-septic agent, and a sitologically acceptable additive for alleviating or preventing sepsis, MODS or septic shock.

In a preferred embodiment, it is the other object of the present invention to provide a health care food comprising the purified HS-23 extract of Lonicera Japonica THUNBERG purified by the above-described method for alleviating or preventing sepsis, MODS or septic shock, together with a sitologically acceptable additive.

In a preferred embodiment, it is the other object of the present invention to provide a health care food comprising the combination of purified HS-23 extract of Lonicera Japonica THUNBERG purified by the above-described method with the commercially available anti-septic agent for alleviating or preventing sepsis, MODS or septic shock, together with a sitologically acceptable additive.

The crude drug composition of inventive health functional food or health care food is used in the form of pulverized form thereof, extracted form therefrom or dried extract form thereof.

The term “a sitologically acceptable additive” disclosed herewith comprises the additive which can be conventionally available well-known in the art, for example food additive lists published on U.S. Food and Drug Administration (See, www.fda.gov/food).

The health functional food composition for preventing and improving purposed diseases could contain about 0.01 to 95 w/w%, preferably 0.5 to 80 w/w% of the above crude extract based on the total weight of the composition.

Above described the crude drug composition therein can be added to food, additive or beverage for prevention and improvement of purposed diseases. For the purpose of preventing and improving purposed diseases, wherein, the amount of above described crude drug composition in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract as an essential component in the indicated ratio, there is no particular limitation on the other liquid component wherein the other component can be various deodorant or natural carbohydrate etc such as conventional beverage. Examples of aforementioned natural carbohydrate are monosaccharide such as glucose, fructose etc; disaccharide such as maltose, sucrose et al.; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartame etc, may be useful favorably. The amount of above described natural carbohydrate generally ranges from about 1 to 20 g, preferably 5 to 12 g in the ratio of 100 ml of present beverage composition.

The other components than aforementioned composition are various nutrients, a vitamin, a mineral or and electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

It will be apparent to those skilled in the art that various modifications and variations can be made in the compositions, use and preparations of the present invention without departing from the spirit or scope of the invention.

### Advantageous Effects of Invention

The present invention provides a method for preparing a purified extract of Lonicera Japonica THUNBERG comprising abundant amount of active ingredients.

The present invention provides a pharmaceutical composition comprising the purified extract of Lonicera Japonica THUNBERG as an active ingredient in an effective amount for preventing and treating sepsis and septic shock.

The present invention also provides a use of above extract for the preparation of pharmaceutical composition to treat and prevent sepsis and septic shock in mammal or human.

### BRIEF DESCRIPTION OF DRAWINGS

The above and other objects, features and other advantages of the present invention will more clearly understood from the following detailed description taken in conjunction with the accompanying drawings, in which;

FIG. 1 shows the HPLC data of CGA(A), HS-23 (b) and SL-101 (c) (* retention time-18 mins (CGA) and 9 & 21 mins-CGA derivatives);

FIG. 2 shows the change of the survival rate in imipenem treatment group and the combined treatment group of imipenem with HS-23 in severe sepsis CLP model.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention provides a method for preparing a purified extract of Lonicera Japonica THUNBERG comprising abundant amount of active ingredients.

The present invention provides a pharmaceutical composition comprising the purified extract of Lonicera Japonica THUNBERG as an active ingredient in an effective amount for preventing and treating sepsis and septic shock.

The present invention also provides a use of above extract for the preparation of pharmaceutical composition to treat and prevent sepsis and septic shock in mammal or human.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Mode for the Invention

### Comparative Example 1

### Preparation of the Conventional Crude Extract of Lonicera Japonica THUNBERG (SL-101)

Dried flower of Lonicera Japonica THUNBERG was added to 10 fold volume of distilled water and extracted by hot-water reflux extraction method at 100° C., for 3 hours. The solution was filtered and the filtrate was concentrated at less than 60° C. using by vacuum evaporator (EYELA N-1000, EYELA Ltd. JAPAN) and dried with lyophilizer (FDCF-12012, Operon Co. Korea) to obtain dried crude flower extract of Lonicera Japonica THUNBERG (Yield: 40.0%. designated as “SL-101” hereinafter). The dried powder was used in following experiments as a comparative test sample.

### Example 1

### Preparation of the Purified Extract of Lonicera Japonica THUNBERG (HS-23a. 23b)

1-1. Use of 5 Fold Weight of HP-20 Resin (HS-23a)

Dried flower of Lonicera Japonica THUNBERG was added to 10 fold volume of distilled water and extracted by hot-water reflux extraction method at 100° C., for 3 hours. The solution was filtered and the filtrate was concentrated at less than 60° C. using by vacuum evaporator (EYELA N-1000, EYELA Ltd. JAPAN) and dried with lyophilizer (FDCF-12012, Operon Co. Korea) to obtain dried crude flower extract of Lonicera Japonica THUNBERG. The extract was suspended in 1.5 fold volume of water (v/w, based on the weight of the crude extract) and the suspension was fractionated with equivalent volume of ethylacetae three times to remove ethylacetate-soluble fraction to afford the 1^{st} purified extract. 5 fold weight of water (w/w, based on the weight of the 1^{st} purified extract) was added to the extract and the equivalent weight of HP-2 resin (Mitsubishi Chemical) was added thereto to stir for 5 hours in order to removing the water-soluble substance. 10 fold volume of water and equivalent amount of 30% methanol was added to the remaining fraction as washing solvent and stirred for 5 hours to afford the 2^{nd} purified extract. The adsorbed adsorbent was concentrated under vaccuo at less than 60° C. using by vacuum evaporator (EYELA N-1000, EYELA Ltd. JAPAN) and dried with lyophilizer (FDCF-12012, Operon Co. Korea) to obtain purified flower extract of Lonicera Japonica THUNBERG (final yield: 3.1%. designated as “HS-23a” hereinafter). The dried powder was used in following experiments as a test sample.

1-2. Use of 10 Fold Weight of HP-20 Resin (HS-23b)

All the purification process was identical with the procedure disclosed in Example 1-1 excepting the used amount of HP-21 resin was 10 fold weight of water (w/w, based on the weight of the 1^{st} purified extract) to obtain purified flower extract of Lonicera Japonica THUNBERG (final yield: 3.1%. designated as “HS-23b” hereinafter). The dried powder was used in following experiments as a test sample.

### Example 2

### Preparation of Purified Extract of Lonicera Japonica THUNBERG (HS-23c)

Dried flower of Lonicera Japonica THUNBERG was added to 10 fold volume of distilled water and extracted by hot-water reflux extraction method at 100° C., for 3 hours. The solution was filtered and the filtrate was concentrated at less than 60° C. using by vacuum evaporator (EYELA N-1000, EYELA Ltd. JAPAN) and dried with lyophilizer (FDCF-12012, Operon Co. Korea) to obtain dried crude flower extract of Lonicera Japonica THUNBERG. The extract was suspended in 1.5 fold volume of water (v/w, based on the weight of the crude extract) and the suspension was fractionated with equivalent volume of ethylacetae three times to remove ethylacetate-soluble fraction to afford the 1^{st} purified extract. 10 fold weight of water (w/w, based on the weight of the 1^{st} purified extract) was added to the extract and the equivalent weight of SP-850 resin (Mitsubishi Chemical) was added thereto to stir for 5 hours in order to removing the water-soluble substance. 10 fold volume of water was added to the remaining fraction as washing solvent with checking the remained water soluble substance. 3 fold volume of 10% methanol was added to the remaining fraction to stir for 5 hours and 3 fold volume of 20% methanol was added to the remaining resin with the similar method to concentrate and dry under vaccuo. The washing procedure was repeated by using 30% methanol and the elute was collected with elutes from the washing procedure using by 10%, 20% and 30% methanol. The collected 2^{nd} purified extract was concentrated under vaccuo at less than 60° C. using by vacuum evaporator (EYELA N-1000, EYELA Ltd. JAPAN) and the concentrates was dissolved in 3 fold volume of 30% methanol. The solution was further purified by using Sephadex LH resin (GE Healthcare, USA) to remove the remaining ineffective ingredients having less than 2.5 kD M.W., such as steroid, terpenoid, lipid, polyphenol, alkaloid, amino acid etc. The remaining elute running with 30% methanol solvent as a mobile phase was concentrated under vaccuo at less than 60° C. using by vacuum evaporator (EYELA N-1000, EYELA Ltd. JAPAN) and dried with lyophilizer (FDCF-12012, Operon Co. Korea) to obtain purified flower extract of Lonicera Japonica THUNBERG (final yield: 1.6%. designated as “HS-23c” hereinafter). The dried powder was used in following experiments as a test sample.

### Example 3

### Preparation of the Purified Extract of Lonicera Japonica THUNBERG (HS-23d)

Dried flower of Lonicera Japonica THUNBERG was added to 10 fold volume of distilled water and extracted by hot-water reflux extraction method at 100° C., for 3 hours. The solution was filtered and the filtrate was concentrated at less than 60° C. using by vacuum evaporator (EYELA N-1000, EYELA Ltd. JAPAN) and dried with lyophilizer (FDCF-12012, Operon Co. Korea) to obtain dried crude flower extract of Lonicera Japonica THUNBERG. The extract was suspended in 1.5 fold volume of water (v/w, based on the weight of the crude extract) and the suspension was fractionated with equivalent volume of ethylacetae three times to remove ethylacetate-soluble fraction to afford the 1^{st} purified extract. 10 fold weight of water (w/w, based on the weight of the 1^{st} purified extract) was added to the extract and the equivalent weight of Amberlite-XAD-2 resin (Rohm & Haas Co., USA) was added thereto to stir for 5 hours in order to removing the water-soluble substance. 10 fold volume of water and equivalent amount of 30% methanol was added to the remaining fraction as washing solvent and stirred for 5 hours to afford the 2^{nd} purified extract. The adsorbed adsorbent was concentrated under vaccuo at less than 60° C. using by vacuum evaporator (EYELA N-1000, EYELA Ltd. JAPAN) and dried with lyophilizer (FDCF-12012, Operon Co. Korea) to obtain purified flower extract of Lonicera Japonica THUNBERG (final yield: 2.8%. designated as “HS-23d” hereinafter). The dried powder was used in following experiments as test sample.

### Example 4

### Component Analysis

The component of the extract prepared in Comparative Example and Examples was analyzed using by HPLC according to the condition disclosed in Table 1 and the result was shown in Table 2.

1-1.Reagent

CGA (chlorogenic acid>96%, Aldrich C3878), TFA (Trifluoroacetic acid, Sigma Aldrich 299537), HP-20 resin (Mitsubishi Chemicals), Acetonitrile (Burdick & Jackson for HPLC), Water (Burdick & Jackson for HPLC), and Ethylacetate (Junsei) were used in the experiment.

1-2.Sample treatment

100 mg of thee extract prepared in Comparative Example and Examples was dissolved in 70 ml of distilled water and extracted by sonication extraction. The extract was filtered and distilled water was added to the filtrate to make 100 ml and the component of each filtrate was analyzed by HPLC to determine the component pattern and the amount of active ingredients in each extract.

TABLE 1
HPLC condition

| Column | Agilent Eclipse Plus C8 HPLC column (4.6 × 250 mm, 5 micrometer) | | | | |
|---|---|---|---|---|---|
| Mobile phase | 0~25 min | 25~30 min | 0~25 min | 0~25 min | 0~25 min |
| (gradient) | 0.1% TFA | 0.1% TFA | 0.1% TFA | 0.1% TFA 0→ | 0.1% TFA |
| | 93 (%)ACN | 93→ 0ACN | 0ACN 100 | 9ACN 100→ 7 | 93ACN 7 |
| | 7 (%) | 7→ 100 | | | |

| | |
|---|---|
| Flow rate | 1.0 ml/min |
| Absorbance | UV 330 nm |
| Peaks | Chlorogenic acid (CGA) |

1-3.Result

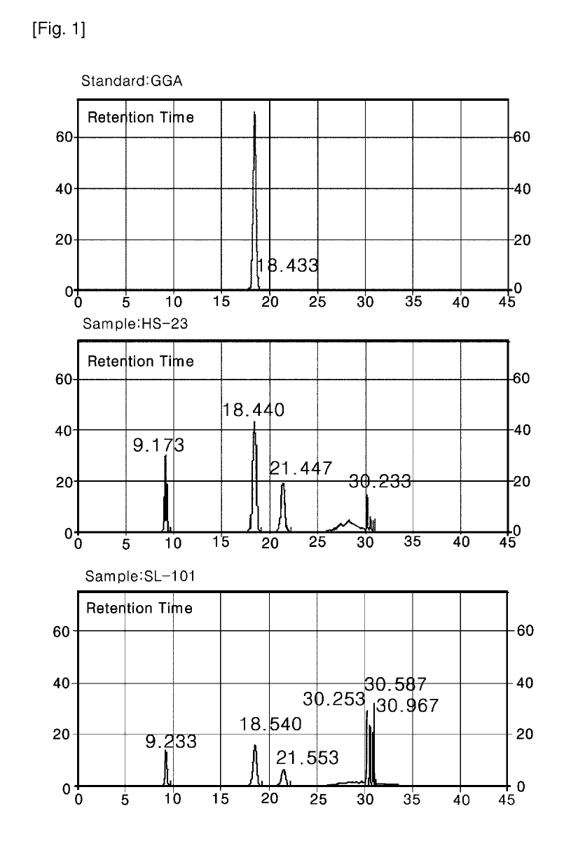

As can be seen in table 2, it has been confirmed that the amount of chlorogenic acid (CGA) and its derivatives, for example, 3,5-O-caffeoylquinic acid, methyl 3,5-di-O-caffeoyl quinate and the like, was sharply increased by about 5.8 fold and about 5.2 fold yield in respect to the yield of chlorogenic acid and the total chlorogenic acid derivatives including chlorogenic acid, respectively, in HS-23b purified extract compared with SL-101 extract prepared according to the conventional extraction method in the art.(See FIG. 1).

TABLE 2
HPLC Result

| | Extract | | | |
|---|---|---|---|---|
| | HS-23a (Use of 10 5 fold | | | |
| | weight of HP-20 resin) | | HS-23b (Use of 10 fold | |
| | Content | | weight of HP-20 resin) | |
| | of A* | Content of B** | Content of A* | Content of B** |
| SL-101 | 2.6% | 3.8% | 1.6% | 2.5% |
| HS-23 | 2.8% | 3.9% | 9.4% | 12.8% |

*A: chlorgenic acid
**B: chlorogenic acid and it's derivative

### Experimental Example 1

### Combined Treatment with Antibiotics in Severe Sepsis CLP Model

To assess the treating activity of the combination of the purified extract prepared in the Examples with antibiotics on severe sepsis, following experiment was performed by using severe sepsis CLP model according to the modified procedure disclosed in the procedure (Daniel Rittirsch, et al. Immuodesign of Experimental sepsis by cecal ligation and puncture. Nature Protocols, Vol. 4(1), pp 31-36, 2009).

Anesthetic(ketamine hydrochloride, 100 mg/kg, Yuhan Pharm. Co.) was intraperitoneally injected to the mice (ICR female, 23-25 g, 8 weeks-old, www.dhbiolink.com) and sepsis was induced to the mice using by CLP (Cecal ligation and puncture). Cavus abdominis medians was dissected and cecum was exposed to ligate the distal ileocecal valve by silk suture (medical suture thread No. 1 & No. 2, www. lead-care.com), Two-holes were made at the cecum by a thread so as to exude the certain amount of fecal material. The cecum was put into the abdominal cavity together with fecal material and physiological saline solution was subcutaneously injected thereto to induce sepsis. The sample prepared in Example (HS-23b) was intravenously injected to the tail.

In particular, the suturing position of the distal ileocecal valve was changed and the number of puncture was more than 2 to increase the amount of fecal material in this experiment in order to inducing severe sepsis CLP model.

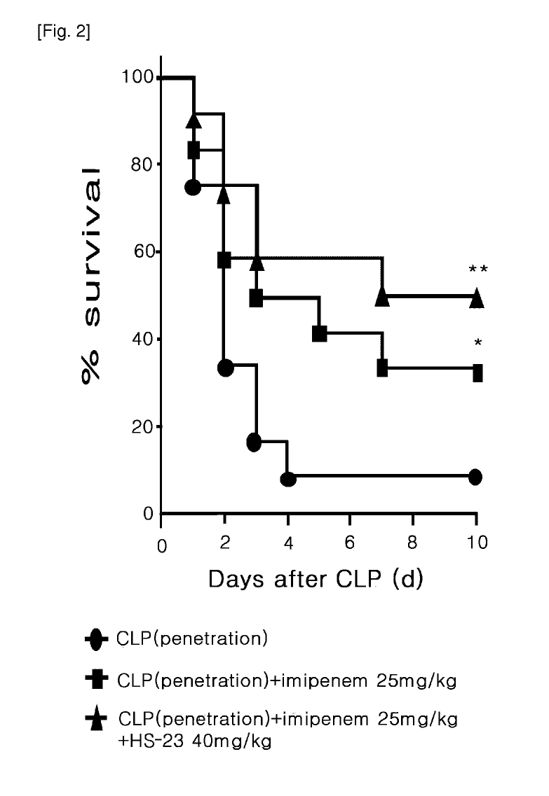

The survival rate in the sole treatment group treated with only HS-23 (40 mg/kg) at O and 24 hours after the induction of severe sepsis CLP model and in the combined treatment group treated with HS-23 (40 mg/kg) and antibiotic(imipenem, Cat. No., 10160, Sigma-Aldrich, 25 mg/kg) at every 12 hours for 4 days was determined for 10 days. The final survival rate at 10th days was found in the CLP treatment group (8%), sole treatment group of imipenam (33%) and combined treatment group of imipenem with HS-34 (50%), respectively, which means that the combined treatmentof imipenem with HS-23 showed increasing effect on the survival rate by 17% compared with the sole treatment group with conventional broad spectrum antibiotics (See FIG. 2).

The survival rate at 1^{st} day, 2^{nd} day, 3^{rd} day and 4^{th} day to 10^{th} day after the CLP in the vehicle group treated only physiological saline solution and PBS (Phosphate buffered saline), was 75%, 33%, 17% and 8%, respectively.

The survival rate at 1^{st} day, 2^{nd} day, 3^{rd} day and 4^{th} day to 10^{th} day after the CLP in the sole treatment group treated only Imipenem (25 mg/kg), was 83%, 58%, 50% and 42%, respectively.

The survival rate at 1^{st} day, 2^{nd} day, 3^{rd} day and 4^{th} day to 10^{th} day after the CLP in the combined treatment group of imipenem (25 mg/kg) with HS-34 (40 mg/kg), was 92%, 75%, 58% and 50%, respectively.

### Experimental Example 2

### Evaluation of Efficacy on MODS in Severe Sepsis CLP Model

To assess the treating activity of the extract of the purified extract prepared in the Examples on MODS in severe sepsis CLP model, followingexperiment was performed by using severe sepsis CLP model according to the modified procedure disclosed in the procedure (Coskun A. K. et al., The effects of montelukast on antioxidant enzymes and pro-inflammatory cytokines on the heart, liver, lungs and kidneys in a rat model of cecal ligation and puncture-induced sepsis, Scientific World Journal, 2011, Vol 11, pp. 1341-1356).

The functional injury of various organs, i.e., liver, kidney, heart etc was observed by H&E12 staining method and the level of respective indicatorfor assessing the function of each organs using by automatic chemical analyzer (Hitachi 7600, Tokyo, JAPAN), i.e., ALT (Alanine Aminotransferase) for liver function; BUN (Blood Urea Nitrogen) and CRE (Creatinine) for kidney function; and LDH (Lactate Dehydrogenase) for heart.

At 1, 3, 6, 12, 24, and 48 hours after the CLP, the blood was collected to isolate serum. The level of ALT, BUN, CRE and LDH was determined and the result was shown in Table 3.

As shown in Table 3, the increased level of ALT, BUN, CRE and LDH was sharply decreased, which means the potent inhibiting effect of test sample on MODS in severe sepsis CLP model.

TABLE 3
Result of MODS test

| | | Test time | | |
|---|---|---|---|---|
| | | 12 hr | 24 hr | 48 hr |
| ALT | CLP | 135.2 ± 19.0 | 197.2 ± 8.0 | 89.4 ± 9.2 |
| (IU/l) | CLP + HS- | 93.5 ± 4.3 | 130.3 ± 13.4 | 75.1 ± 9.1 |
| | 23 | | | |
| BUN | CLP | 37.9 ± 2.2 | 32.0 ± 4.4 | 25.4 ± 1.7 |
| (mg/dl) | CLP + HS- | 26.2 ± 2.1 | 22.1 ± 1.4 | 17.2 ± 1.5 |
| | 23 | | | |
| CRE | CLP | 0.26 ± 0.02 | 0.30 ± 0.03 | 0.16 ± 0.01 |
| (mg/dl) | CLP + HS- | 0.17 ± 0.02 | 0.21 ± 0.01 | 0.16 ± 0.02 |
| | 23 | | | |
| LDH | CLP | — | 1564.3 ± 98.1 | 977.3 ± 111.0 |
| (IU/l) | CLP + HS- | 1056.5 ± 105.6 | 1237.0 ± 61.8 | — |
| | 23 | | | |

### Experimental Example 3

### Determination of Survival Rate and Efficacy in LPS-Induced Inflammation Model

To assess the survival rate and efficacy of the extract of the purified extract prepared in the Examples in LPS-induced inflammation model, following experiment was performed according to the modified procedure disclosed in the procedure (Masami Yamada et al., Discovery of Novel and Potent Small Molecule Inhibitors of NO and cytokine Production as Antisepsis Agents: Synthesis and Biological Activity of Alkyl 6-(N-substituted sulfamoyl)cyclohex-1-ene-1-carboxylate, J. Med. Chem. 2005, 48, pp. 7457-7467).

LPS (Lipopolysaccharide; E. Coli 0111:B4 Sigma-Aldrich, L4130) was intraperitoneally injected into mice (C57BL/6, female, 8 weeks-old, KRIBB) and 1-8 hours after the injection, HS-23 was intraveneously injected to determine the survival rate of mice.

The survival rate in the group treated with HS-23 at one hour after LPS injection, showed 90% at 18 hours, 80% at 21 hours and 70% at 24-72 hours after LPS injection (See Table 4).

The expressed level of various pro-inflammatory factors in the serum isolated from blood by centrifugation wherein the blood had been collected at every hour after LPS injection was determined by ELISA method and the result was shown in Table 5 to Table 8.

Through the determination at every hour, the expressed level of TNF-alpha, IL-1beta and IFN-gamma and HMGB-1 was found to reach to maximum level at 1 hour, 8 hours and 19 hours after the LPS injection.

TABLE 4
Result of survival rate test in LPS inflammation model

| | Hours after LPS injection | | | | |
|---|---|---|---|---|---|
| | 18 hrs | 21 hrs | 24 hrs | 48 hrs | 72 hrs |
| LPS injection | 80% | 50% | 30% | 10% | 0% |
| Treatment of HS-23 | 90% | 80% | 70% | 70% | 70% |
| (3 mg/kg) at 1 hr after | | | | | |
| LPS injection | | | | | |
| Treatment of HS-23 | 80% | 50% | 50% | 20% | 10% |
| (3 mg/kg) at 4 hr after | | | | | |
| LPS injection | | | | | |
| Treatment of HS-23 | 80% | 50% | 40% | 20% | 10% |
| (3 mg/kg) at 8 hr after | | | | | |
| LPS injection | | | | | |

TABLE 5
Change of the TNF-alpha level after LPS injection

| | Hours after LPS injection | | | | |
|---|---|---|---|---|---|
| | normal | 1 hr | 2 hr | 4 hr | 6 hr |
| TNF-alpha (pg/ml) | 63 | 7,715 | 2,485 | 1,057 | 710 |

TABLE 6
Change of the IL-1beta level after LPS injection

Hours after LPS injection

| | normal | 4 hr | 8 hr | 12 hr |
|---|---|---|---|---|
| IL-1beta (pg/ml) | 46 | 288 | 1,341 | 1,177 |

TABLE 7
Change of the IFN-gamma level after LPS injection

Hours after LPS injection

| | Normal | 4 hr | 8 hr | 12 hr |
|---|---|---|---|---|
| IFN-gamma (pg/ml) | 150 | 1,472 | 29,886 | 18,283 |

TABLE 8
Change of the HMGB-1 level after LPS injection

| | Hours after LPS injection | | | | |
|---|---|---|---|---|---|
| Hours after LPS injection | normal | 1 hr | 2 hr | 4 hr | 6 hr |

| | | | | | |
|---|---|---|---|---|---|
| HMGB-1 (pg/ml) | 787 | 1,227 | 5,719 | 8,818 | 3,900 |

LPS was intraperitoneally injected into the mice simultaneously with the intravenous injection of HS-23 extract and the time at the level of pro-inflammatory factors, i.e., TNF-alpha, IL-1beta and IFN-gamma and HMGB-1 reached to maximum level, was determined according to ELISA method.

At the result, it has been confirmed that the HS-23 treatment group strongly inhibited the increased level of expressed pro-inflammatory factors as shown in Table 9.

TABLE 9
Effect on the level of Pro-inflammatory Factors.

| | | Group | | | |
|---|---|---|---|---|---|
| | | | | LPS + | LPS + |
| | | | | HS-23 | HS-23 |
| | | vehicle | LPS | (1 mg/kg) | (3 mg/kg) |
| TNF-alpha | 1 hr after | 60 | 7,715 | 2,951* | 1,940* |
| (pg/ml) | | | | | |
| IL-1beta | 8 hrs after | 20 | 1,341 | 823* | 128* |
| (pg/ml) | | | | | |
| IFN-gamma | 8 hrs after | 206 | 29,886 | 28,576 | 476* |
| (pg/ml) | | | | | |
| HMGB-1 | 19 hrs after | 1,122 | 8,818 | 7,863 | 1,806* |
| (pg/ml) | | | | | |

*p < 0.05 vs. LPS alone-challenged group

### Experimental Example 4

### Stability Test

To confirm the stability of the extract of the extract of the purified extract prepared in the Examples and the injection preparation containing the lyophilized form of HS-23 extract, following experiment was performed according to the stability guideline of Korea Food & Drug Administration (www.kfda.go.kr).

4-1. Test Procedure.

The test material and the injection preparation containing the lyophilized form of HS-23 extract were subject to long-term stability test according to the stipulation and guideline of Korea Pharmacopoeia at refrigerator (5±3° C.) for 2 years.

4-2. Test Result

As shown in Table 10 and 11, it has been confirmed that the amount of standard substance of Lonicera Japonica THUNBERG, i.e., chlorogenic acid(CGA), has not changed during the stability test period and the test sample was very pharmaceutically stable enough to be passed to the guideline.

TABLE 10
stability test of HS-23 extract

| | | | Heavy | Loss on | Amount of |
|---|---|---|---|---|---|
| | Sensory | Identification | Metal | drying | CGA in |
| Months | test | test | limit test | test | sample (mg/g) |
| 0 | Passed | Passed | Passed | 1.6% | 62.0 |
| 3 | Passed | Passed | Passed | 1.6% | 61.7 |
| 6 | Passed | Passed | Passed | 1.6% | 61.5 |
| 9 | Passed | Passed | Passed | 1.6% | 61.3 |
| 12 | Passed | Passed | Passed | 1.6% | 61.0 |
| 18 | Passed | Passed | Passed | 1.6% | 60.8 |
| 24 | Passed | Passed | Passed | 1.6% | 60.4 |

TABLE 11
stability test of injection preparation containing HS-23 extract

| | | | Heavy | | Amount | | | |
|---|---|---|---|---|---|---|---|---|
| | Sensory | Identification | Metal limit | pH | of CGA in | Sterility | Insoluble | Particulate |
| Month | test | test | test | test | sample (%) | test | particletest | matter test |
| 0 | passed | passed | passed | 5.3 | 110 | negative | passed | passed |
| 3 | passed | passed | passed | 5.3 | 108 | negative | passed | passed |
| 6 | passed | passed | passed | 5.2 | 108 | negative | passed | passed |
| 9 | passed | passed | passed | 5.3 | 107 | negative | passed | passed |
| 12 | passed | passed | passed | 5.3 | 106 | negative | passed | passed |
| 24 | passed | passed | passed | 5.3 | 105 | negative | passed | passed |

### Experimental Example 5

### Toxicity Test

To confirm the toxicity of the extract of the extract of the purified extract prepared in the Examples, following experiment was performed according to the toxicity guideline of Korea Food & Drug Administration (www.kfda.go.kr).

All the toxicity tests including single-dose toxicity test in rodents/non-rodents, safety pharmacology test, genotoxicity test, 4 week's repeated-dose toxicity test etc were performed in GLP certificated company (www.biotoxtech.com).

As can be seen in Table 12, it has been confirmed that the lethal dose of the test sample is more than 500 mg/kg in single-dose toxicitytest and the value of NOAEL is more than 75 mg/kg in 3 week's repeated-dose toxicitytest. Furthermore, the test sample has been proved to be safe at over 100 mg/kg and 300 mg/kg in safety pharmacology test (in cardiovascular system, respiratory system, CNS system, etc).

Accordingly, the inventive purified extract has been proved to be safe and non-toxic.

TABLE 12
toxicity test of HS-23 extract

| Test content | Test result |
|---|---|
| Single-dose toxicity test | LD(lethal dose): >500 mg/kg (in male), |
| (rat) | >1000 mg/kg (in female) |
| Single-dose toxicity test | MTD(maximum tolerated dose): |
| (beagle dog) | >200 mg/kg |
| 4 week's repeated dose | NOAEL(No-observed-adverse-effect |
| toxicity test (rat) | level in rat): >75 mg/kg-The increased |
| | weight of spleen was observed but |
| | recovered in 2 week's recovery test |
| 4 week's repeated dose | NOAEL(No-observed-adverse-effect |
| toxicity test (beagle | level in Beagle dog): |
| dog) & 2 week's | 100 mg/kg-MTD(maximum tolerated |
| recovery test | dose): >200 mg/kg |
| Genotoxicity test | reverse mutation test: negative- |
| | chromosome aberration test: negative- |
| | mouse micronucleus test: negative |
| Safety pharmacology test | Cardiovascular system: no effect on |
| | blood pressure, heart rate, electrocardiography |
| | etc at the dose of 6.25, 35 and |
| | 100 mg/kg-Respiratory system: no effect |
| | on respiratory rate/min, respiratory |
| | volume/min, tidal volume etc at the dose |
| | of 75,150 and 300 mg/kg-CNS: no effect |
| | on motility, behavior change, behavior co- |
| | operation, body temperature, reflex on |
| | sensory/motor nerve etc at the dose of |
| | 75,150 and 300 mg/kg |

Hereinafter, the formulating methods and kinds of excipients will be described, but the present invention is not limited to them. The representative preparation examples were described as follows.

Preparation of Powder

Dried powder of HS-23b 20 mg

Lactose 100 mg

Talc 10 mg

Powder preparation was prepared by mixing above components and filling sealed package.

Preparation of Tablet

Dried powder of HS-23a 10 mg

Corn Starch 100 mg

Lactose 100 mg

Magnesium Stearate 2 mg

Tablet preparation was prepared by mixing above components and entabletting.

Preparation of Capsule

Dried powder of HS-23c 10 mg

Corn starch 100 mg

Lactose 100 mg

Magnesium Stearate 2 mg

Capsule preparation was prepared by mixing above components and filling gelatin capsule by conventional gelatin preparation method.

Preparation of Injection

Dried powder of HS-23d 10 mg

Mannitol 180 mg

Distilled water for injection 2974 mg

PH controller (Na₂HPO₄12H₂O) optimum amount

Injection preparation was prepared by dissolving active component, controlling pH to about 7.5 and then filling all the components in 2m1 ample and sterilizing by conventional injection preparation method.

Preparation of Liquid

Dried powder of HS-23b 20 mg

Isomerized sugar 10 g

Mannitol 5 g

Distilled water optimum amount

Liquid medicine was prepared by dissolving the components to distilled water with a proper dose of lemon scent, mixing, adjusting to 100 ml with distilled water in brown bottle and sterilizing by conventional liquid medicine preparation method.

Preparation of Health Functional Food

Dried powder of HS-23a 1000 mg

Vitamin mixture optimum amount

Vitamin A acetate 70 μg

Vitamin E 1.0 mg

Vitamin B₁ 0.13 mg

Vitamin B₂ 0.15 mg

Vitamin B₆ 0.5 mg

Vitamin B₁₂ 0.2 μg

Vitamin C 10 mg

Biotin 10 μg

Amide nicotinic acid 1.7 mg

Folic acid 50 μg

Calcium pantothenic acid 0.5 mg

Mineral mixture optimum amount

Ferrous sulfate 1.75 mg

Zinc oxide 0.82 mg

Magnesium carbonate 25.3 mg

Monopotassium phosphate 15 mg

Dicalcium phosphate 55 mg

Potassium citrate 100 mg

Magnesium chloride 24.8 mg

The above-mentioned vitamin and mineral mixture may be varied in many ways.

Such variations are not to be regarded as a departure from the spirit and scope of the present invention.

Preparation of Health Beverage

Dried powder of HS-23c 100 mg

Citric acid 1000 mg

Oligosaccharide 100 g

Apricot concentration 2 g

Taurine 1 g

Distilled water 900 ml

Health beverage preparation was prepared by dissolving active component, mixing, stirring at 85° C. for 1 hour, filtering and then filling all the components in 2 container and sterilizing by conventional health beverage preparation method.

The invention being thus described, may be varied in many ways. Such variations are not to be regarded as a departure from the spirit and scope of the present invention, and all such modifications as would be obvious to one skilled in the art are intended to be included within the scope of the following claims.

The inventions are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the purified extract of Lonicera Japonica THUNBERG showed potent ant-sepsis activity in severe sepsis CLP model test, the effect on MODS, and the inhibitory effect on various pro-inflammatory cytokines such as TNF-alpha, IL-1beta, IFN-gamma, HMGB-1 etc, as well as it showed unexpectedly synergistic effect on the treatment of sepsis and septic shock in case of combining with the commercially available anti-septic agent such as broad-spectrum anti-biotic to the person skilled in the art,therefore, it can be useful in treating and preventing the sepsis and septic shock as a medicament and health functional food.

1. A method for preparing a purified extract of Lonicera Japonica THUNBERG comprising the step consisting of; extracting the dried flower material of Lonicera Japonica THUNBERG with extracting solvent at 1^{st} step; subjecting the crude extract to at least one treatment selected from filtration method, centrifugation or the combination thereof to afford the crude extract of Lonicera Japonica THUNBERG at 2^{nd} step; suspending the crude extracting water by adding water to prepare the suspended solution and fractionating the solution into non-polar solvent soluble fraction and polar solvent soluble fraction to remove the non-polar soluble substance and to afford the 1^{st} purified extract by collecting the residue at 3^{rd} step; adding water to the 1^{st} purified extract to subject to at least one purification process selected from adsorption chromatography, ion column chromatography or the combination thereof using by the equivalent amount of adsorbent resin to that of water, and washing with washing solvent repeatedly to afford the 2^{nd} purified extract of Lonicera Japonica THUNBERG at 4^{th} step; and concentrating the extract under vaccuo and drying to afford the purified extract of Lonicera Japonica THUNBERG containing abundant amount of active ingredients.

2. The method according to claim 1 wherein said extracting solvent at 1^{st} step comprises 1 to 100 fold volume of at least one solvent based on the weight of flower material (v/w) selected from the group consisting of water, spirit, methanol, ethanol, propanol, butanol, hexane, ethylacetate, cyclohexane, DMSO, chloroform and methylene chloride.

3. The method according to claim 1 wherein said extracting solvent at 1^{st} step comprises 1 to 100 fold volume of basic solution dissolving weak base such as NaHCO₃, NaCO₃ etc in an amount of 0.1 to 5% weight based on the weight of the flower material (w/w) in water to improve the extraction efficiency.

4. The method according to claim 1 wherein said extracting process at 1^{st} step is performed by at least one extraction method selected from hot-water reflux extraction, enfleurage extraction, Soxhlet extraction, sonication extraction and the combination thereof.

5. The method according to claim 1 wherein said treatment to afford the crude extract of Lonicera Japonica THUNBERG at 2^{nd} step is performed by at least one treatment selected from filtration method, centrifugation or the combination thereof.

6. The method according to claim 1 wherein said process to afford the 1^{st} purified extract process at 3^{rd} step is performed by adding about 0.005 to 5 fold volume of water (v/w, based on the weight of the crude extract) to prepare the suspended solution; and fractionating the solution into non-polar solvent soluble fraction and polar solvent soluble fraction to remove the non-polar soluble substance by adding about 0.1 to 50 fold volume of non-polar solvent (v/v, based on the volume of the suspension) such hexane, methylene chloride, chloroform, or ethyl acetate.

7. The method according to claim 1 wherein said process to afford the 2^{nd} purified extract of Lonicera Japonica THUNBERG at 4^{th} step is performed by adding about 1 to 30 fold weight of water (w/w, based on the weight of the 1^{st} purified extract) to the 1^{st} purified extract to subject to adsorption chromatography by using at least one resin selected from SP207, HP20SS, Diaion HP 20, SP-850 resin, active carbon, or Amberlite XAD-2,4 and the equivalent amount of adsorbent resin to that of water for further purification.

8. The method according to claim 1 wherein said process to afford the 2^{nd} purified extract of Lonicera Japonica THUNBERG at 4^{th} step is performed by subjecting to ion column chromatography using by at least one strongly acidic resin selected from AG 50W-x8, Amberlite IR-120, Amberlite IRA-400, Dowex 50W-x8 or SK1B; at least one weakly acidic resin selected from Amberlite IRC-50, Bio-Rex 70, Duolite-436 or WK40; or at least one weakly basic resin selected from Amberlite IR-67 or Dowex 3-x4, preferably, at least one strongly acidic resin selected from Amberlite IR-120, Amberlite IRA-400 or SK1B and the equivalent amount of ionic resin to that of water.

9. The method according to claim 1 wherein said washing process to afford the 2^{nd} purified extract of Lonicera Japonica THUNBERG at 4^{th} step is performed by washing the adsorbent to the resin with at least on washing solvent selected from water, methanol, ethanol, propanol, butanol or the mixture thereof repeatedly.

10. The method according to claim 1 wherein said washing process to afford the 2^{nd} purified extract of Lonicera Japonica THUNBERG at 4^{th} step is performed to further sephadex column chromatography using by at least on sephadex resin selected from Sephadex LH-20 resin, Sephadex G15 resin or Sephadex G35 besides the purification process at 4^{th} step.

11. The method according to claim 1 wherein said concentrating process and drying process to afford the purified extract of Lonicera Japonica THUNBERG at 5^{th} step is performed by concentrating the extract under vaccuo at the temperature ranging from 10 to 80° C. and drying the extract by at least one drying method selected from room temperature drying method, freeze drying method, hot-air drying method or the combination thereof to afford inventive purified extract of extract of Lonicera Japonica THUNBERG.

12. A purified HS-23 extract of Lonicera Japonica THUNBERG containing chlorogenic acid and the derivatives thereof in an amount ranging from 2.0 to 30.0%(w/w) based on the weight of the dried purified extract, which is prepared by the step consisting of; extracting the dried flower material of Lonicera Japonica THUNBERG with extracting solvent at 1^{st} step; subjecting the crude extract to at least one treatment selected from filtration method, centrifugation or the combination thereof to afford the crude extract of Lonicera Japonica THUNBERG at 2^{nd} step; suspending the crude extracting water by adding water to prepare the suspended solution and fractionating the solution into non-polar solvent soluble fraction and polar solvent soluble fraction to remove the non-polar soluble substance and to afford the 1^{st} purified extract by collecting the residue at 3^{rd} step; adding water to the 1^{st} purified extract to subject to at least one purification process selected from adsorption chromatography, ion column chromatography or the combination thereof using by the equivalent amount of adsorbent resin to that of water, and washing with washing solvent repeatedly to afford the 2^{nd} purified extract of Lonicera Japonica THUNBERG at 4^{th} step; and concentrating the extract under vaccuo and drying to afford the purified extract of Lonicera Japonica THUNBERG for the treatment and prevention of sepsis, MODS or septic shock.

13. A pharmaceutical composition comprising the purified HS-23 extract of Lonicera Japonica THUNBERG prepared by the method as set forth in claim 1 as an active ingredient for the treatment and prevention of sepsis, MODS or septic shock.

14. The pharmaceutical composition according to claim 13 wherein said sepsis is mild sepsis, severe sepsis, infection symptoms or sepsis caused by burn, acute laryngopharyngitis, ulcerative colitis, IBS (Irritable Bowel syndrome), rheumatic arthritis, degenerative arthritis, acute hepatitis, or chronic hepatitis.

15. The pharmaceutical composition according to claim 13 wherein said MODS occurs in the injured organ selected from liver, kidney, heart, lung, small intestine, large intestine, duodenum, stomach, pancreas, spleen caused by mild sepsis, severe sepsis, or infection symptoms or sepsis caused by burn.

16. The pharmaceutical composition according to claim 13 wherein said septic shock is septic shock caused by mild sepsis, severe sepsis, infection symptoms or sepsis caused by burn.

17. A pharmaceutical composition comprising the combination of purified HS-23 extract of Lonicera Japonica THUNBERG prepared by the method as set forth in claim 1 with the commercially available antiseptic agent as an active ingredient for the treatment and prevention of sepsis, MODS or septic shock.

18. The pharmaceutical composition according to claim 17 wherein said combination is the combination of (a) purified HS extract of Lonicera Japonica THUNBERG purified by the method as set forth in claim 1 with (b) the commercially available anti-septic agent mixed with the mixed ratio ranging from 0.1˜10:0.1˜10 by weight (w/w).

19. The pharmaceutical composition according to claim 17 wherein said commercially available anti-septic agent is at least one anti-septic agent selected from the group consisting of antibiotics such as penicillin, quinolone, monobactam, aminoglycoside, cephalosporin, tetracycline, glycopeptides, carbapenem and the like; anti-inflammatory agents such as mefenamic acid, indomethacin, ibuprofen, piroxicam, diclofenac and the like; anti-fungal agent such as amphotericin, B, nystatin, griseofulvin, azole anti-fungal agent and the like; and anti-allergic agent such as cetirizine, fexofenadine, chlroropeniramine, and the like.

20. A health functional food comprising purified HS-23 extract of Lonicera Japonica THUNBERG purified by the method as set forth in claim 1 as an active ingredient for alleviating or preventing sepsis, MODS or septic shock.

21. A health functional food comprising the combination of purified HS-23 extract of Lonicera Japonica THUNBERG purified by the method as set forth in claim 1 with the commercially available anti-septic agent as an active ingredient for alleviating or preventing sepsis, MODS or septic shock.

22. A health care food comprising the purified HS-23 extract of Lonicera Japonica THUNBERG purified by the method as set forth in claim 1 for alleviating or preventing sepsis, MODS or septic shock, together with a sitologically acceptable additive.

23. A health care food comprising the combination of purified HS-23 extract of Lonicera Japonica THUNBERG purified by the method as set forth in claim 1 with the commercially available anti-septic agent for alleviating or preventing sepsis, MODS or septic shock, together with a sitologically acceptable additive.

24. A use of the purified HS-23 extract of Lonicera Japonica THUNBERG purified by prepared by the method as set forth in claim 1 for the preparation of therapeutic agent for the treatment and prevention of sepsis, MODS or septic shock in mammal including human.

25. A use of the combination of purified HS-23 extract of Lonicera Japonica THUNBERG purified by the method as set forth in claim 1 with the commercially available anti-septic agent for the preparation of therapeutic agent for the treatment and prevention of sepsis, MODS or septic shock in mammal including human.

26. A method of treating or preventing sepsis, MODS or septic shock in mammal including human comprising administering an effective amount of the purified HS extract of Lonicera Japonica THUNBERG purified by the method as set forth in claim 1, together with a pharmaceutically acceptable carrier thereof to said mammal.

27. A method of treating or preventing sepsis, MODS or septic shock in mammal including human comprising administering an effective amount of the combination of purified HS-23 extract of Lonicera Japonica THUNBERG purified by the method as set forth in claim 1, with the commercially available anti-septic agent, together with a pharmaceutically acceptable carrier thereof to said mammal.
